(19) United States
(12) Patent Application Publication
(10) Pub. No.: US 2025/0381131 A1
(43) Pub. Date: Dec. 18, 2025

(54) COMPOSITION AND METHOD FOR INHIBITING ELASTASE ACTIVITY

(71) Applicant: ACCESS BUSINESS GROUP INTERNATIONAL LLC, Ada, MI (US)

(72) Inventors: Huiji ZHOU, Shanghai (CN); Bo LI, Shanghai (CN); Tingzhao LI, Shanghai (CN); Xiuyu JIANG, Shanghai (CN)

(21) Appl. No.: 19/108,546

(22) PCT Filed: Aug. 31, 2023
(86) PCT No.: PCT/US2023/031779

§ 371 Date: Mar. 4, 2025

(30) Foreign Application Priority Data
Sep. 5, 2022 (CN) 202211078986.5

(51) Int. Cl. A61K 8/9789; A61K 8/9794; A61Q 19/08

(52) U.S. Cl. CPC A61K 8/9789; A61K 8/9794; A61Q 19/08

(57) ABSTRACT

A composition for administration to a subject is disclosed. The composition comprises at least one botanical active component. The botanical active component is present in the composition in an amount effective to inhibit elastase activity in the skin of the subject. The botanical active component comprises at least one extract selected from the group consisting of: i) an extract of Potentilla glabra leaf; ii) an extract of Bombax malabaricum flower; iii) an extract of Amomum villosum seed; iv) an extract of Amomum kravanh fruit; v) an extract of Cornus officinalis fruit; and vi) combinations of i) to v). The composition may be in the form of a topical composition, e.g., for topical application. The composition may also be in the form of an oral composition, e.g., for ingestion. A method of inhibiting elastase activity in the skin of a subject is also provided. The method comprises administering an effective amount of the composition to the subject.

### CROSS-REFERENCE TO RELATED APPLICATIONS

This application claims priority to and all advantages of Chinese Application No. 202211078986.5 filed on 5 Sep. 2022, the contents of which is incorporated herein by reference in its entirety.

### FIELD OF THE INVENTION

The present invention relates generally to compositions and methods for inhibiting elastase activity and, more specifically, to a botanical extract-containing composition for inhibiting elastase activity in skin of a subject. Related methods are also provided.

### BACKGROUND OF THE INVENTION

Elastin, with its inherent resiliency, is vital for providing elasticity to organs, connective tissue, and the skin. Various elastases have been implicated in the breakdown of elastin, i.e. elastic fibers, in various types of tissues during inflammation or diseases as well as during aging. Cumulative exposure to the sun or to UV irradiation causes the formation of wrinkles which are associated with marked decreases in skin elasticity. In skin, elastase is mainly responsible for the degradation of elastin, which is associated with wrinkle formation.

Recently, bioactive molecules from plants have been widely used as cosmeceutical ingredients because of their prime property to slow down the rate of intrinsic skin aging processes and diverged the extrinsic ones. Several natural products and extracts have been reported for the development of anti-aging skin care products. For instance, polyphenols from green tea such as epigallocatechin gallate (EGCG) has been formulated for anti-aging skin care products that exhibit restrained elastase inhibition.

In view of the foregoing, there remains an opportunity to provide new and useful elastase inhibitors. Such inhibitors can offer potential preventive and therapeutic approaches for reducing skin aging, such as wrinkle formation, caused by elastase. As such, there also remains an opportunity to provide new and useful compositions and methods for inhibiting elastase activity.

### BRIEF SUMMARY OF THE INVENTION

A composition for administration to a subject is provided. The composition comprises at least one botanical active component. The botanical active component is generally present in the composition in an amount effective to inhibit elastase in the skin of the subject. The botanical active component comprises at least one extract selected from the group consisting of: i) an extract of Potentilla glabra leaf; ii) an extract of Bombax malabaricum flower; iii) an extract of Amomum villosum seed; iv) an extract of Amomum kravanh seed; v) an extract of Cornus officinalis; and vi) combinations of i) to v).

In various embodiments, the composition is a topical composition that is formulated for topical administration to the subject. In other embodiments, the composition is an oral composition that is formulated for oral administration to the subject.

The composition can be used for inhibiting elastase in the skin of a subject. A method of inhibiting elastase in the skin of a subject comprises administering an effective amount of the composition to the subject.

### BRIEF DESCRIPTION OF THE DRAWINGS

FIG. 1 is a plot showing the average inhibition rate of elastase using EGCG as a control;

FIG. 2 is a plot showing the average inhibition rate of elastase using Potentilla glabra Lodd. var. mandshurica (Maxim.) Hand.-Mazz (leaf)—ethanol extract;

FIG. 3 is a plot showing the average inhibition rate of elastase using Bombax malabaricum DC. (flower)—ethanol extract;

FIG. 4 is a plot showing the average inhibition rate of elastase using Amomum villosum Lour (seed)—ethanol extract;

FIG. 5 is a plot showing the inhibition rate of elastase using Amomum kravanh Pierre ex Gagnep (fruit)—ethanol extract;

FIG. 6 is a plot showing the average inhibition rate of elastase using Cornus officinalis Sieb.et Zucc. (fruit)—aqueous extract; and

FIG. 7 is a plot showing the average inhibition rate of elastase using Cornus officinalis Sieb.et Zucc. (fruit)—ethanol extract.

### DETAILED DESCRIPTION OF THE CURRENT EMBODIMENTS

A composition for administration to a subject is disclosed. The composition comprises at least one botanical active component. The composition is described below, followed by description of associated uses and methods.

As will be understood in view of this disclosure, the composition is not particularly limited aside from the botanical active component and, in particular, the extract(s) thereof, as well as the related components and methods. As such, the composition may be formulated, for example, as a topical composition (e.g., a cosmetic composition), or as an oral composition or as a nutraceutical, pharmaceutical, or supplement, and may be utilized as a unique and stand-alone therapeutic or in combination with other therapeutics compatible therewith.

The composition is useful for treating, preventing, and/or ameliorating various conditions, such as those associated with aging. Specifically, as will be appreciated in view of the description and examples below, the composition of the present embodiments is believed to be capable of inhibiting elastase activity and, more specifically, inhibiting elastin degradation caused by elastase in the skin of a subject.

As such, the composition may be utilized to treat (i.e., slow, prevent, reverse, etc.) age-related or other conditions generally associated with elastase activity, such as the formation of wrinkles.

As introduced above, the composition comprises a botanical active component. More specifically, the botanical active component comprises, optionally consists essentially of, or optionally consists of at least one extract selected from the group consisting of: i) an extract of Potentilla glabra leaf; ii) an extract of Bombax malabaricum flower; iii) an extract of Amomum villosum seed; iv) an extract of Amomum kravanh seed; v) an extract of Cornus officinalis fruit, and vi) combinations of i) to v). In certain embodiments, the composition of this disclosure is free of other active ingredients. By “other active ingredients”, it is generally meant that the composition is free of other types of Traditional Chinese Medicines (“TCMs”; or “Chinese medicines”) that are different from the extracts above and exemplified below. Other types of TCMs are understood in the art.

In certain embodiments, only one of the five extracts i) to v) is present in the composition. In other embodiments, only two of the five extracts i) to v) is present in the composition. In yet other embodiments, only three of the five extracts i) to v) is present in the composition. In yet other embodiments, only four of the five extracts i) to v) is present in the composition. In yet other embodiments, all five of the extracts i) to v) are present in the composition. Each of the individual extracts i) to v) may be referred to simply as the “botanical extract” or collectively as the “botanical extracts” and are described in turn below.

The term “extract” is used herein in the conventional sense to refer to a composition that has been obtained via fluid extraction from a source material. As such, the term “botanical extract” is to be understood as a composition obtained via fluid extraction (e.g., solvent extraction, gas extraction, CO₂ extraction, etc.) from a botanical source (i.e., a plant material). Botanical extracts suitable for use in the composition can be obtained via any extraction method, or combination of such methods, known in the art, including water extractions, steam extractions, solvent extractions, etc. Exemplary extraction techniques are described below. However, the botanical extracts are generally not limited to a particular extraction method, or additional/adjuvant techniques used to obtain the botanical extracts, but rather may vary according to the parameters described herein. Additionally, an extraction step is not required to prepare the botanical active component and/or the composition, as suitable extracts (e.g. standardized extracts) are readily available from a number of commercial suppliers.

Botanical extracts suitable for use in, or as, the botanical active component include those obtained via solvent extraction, e.g. via use of a polar solvent such as an alcohol (e.g. methanol, ethanol, butylene glycol, etc.), ether (e.g. diethyl ether, methyl tert butyl ether, etc.), ketone (e.g. acetone), ester (e.g. ethyl acetate), phenol, water, and the like, a nonpolar solvent such as benzene, xylenes, toluene, etc., as well as derivatives, modifications, and combinations thereof (e.g. solvent-water blends, including alcohol-water, acetone-water, etc.). Additional and alternative extraction techniques include sequential fractionations, total hydro-ethanolic extractions, lump-sum extractions, supercritical fluid extractions (e.g. with CO₂), and the like, as well as those utilizing sequential or secondary extractions from a first extract (e.g. a non-polar solvent extract of a botanical extract obtained from a polar solvent extraction) or other processing techniques such as filtration, purification, distillation, dehydration, evaporation, concentration, drying, etc. Specific examples of suitable extraction methods are described in U.S. Pat. No. 7,897,184, which is incorporated by reference herein.

As understood in the art, various sections or parts of plants can be used to obtain the essential oils and extracts, such as bark, berries, flowers, fruits, leaves, peels, resins, rhizomes, roots, seeds, and/or woods. Essential oils can be obtained by a number of processes, such as by distillation (e.g. using steam), expression, solvent extraction, absolute oil extraction, resin tapping, and/or cold pressing.

In various embodiments, the solvent used to obtain suitable botanical extracts for this disclosure is one in which the resulting botanical extract and/or a subsequent form thereof (e.g. botanical extract powder) is suitable for ingestion. For example, the solvent is water or ethanol.

In one example, the botanical extracts can be obtained using an organic solvent extraction technique. In another example, solvent sequential fractionation can be used to obtain the botanical extracts. Total hydro-ethanolic extraction techniques can also be used to obtain the botanical extracts. Generally, this is referred to as a lump-sum extraction. The botanical extract generated in the process will contain a broad variety of phytochemicals present in the extracted material including fat and water-soluble phytochemicals. Following collection of the botanical extract solution, the solvent will be evaporated, resulting in the botanical extract.

Total ethanol extraction may also be used. This technique uses ethanol as the solvent. This extraction technique generates a botanical extract that may include fat soluble and/or lipophilic compounds in addition to water-soluble compounds. Total methanol extraction may also be used in a similar manner with similar results.

Another example of an extraction technique that can be used to obtain the botanical extracts is supercritical fluid carbon dioxide extraction (SFE). In this extraction procedure, the material to be extracted is not exposed to any organic solvents. Rather, the extraction solvent is carbon dioxide (CO₂), with or without a modifier, in super-critical conditions (e.g. >31.3° C. and >73.8 bar). Those of skill in the art will appreciate that temperature and pressure conditions can be varied to obtain the best yield of botanical extract. This technique generates a botanical extract of fat soluble and/or lipophilic compounds, similar to total hexane and ethyl acetate extraction techniques, which may also be used.

Each of the extraction methods above also may include and/or be utilized in combination with one or more additional processing steps understood in the art. For example, plant material may be comminuted, smashed, ground, etc. There also may be one or more filtration steps to remove, for example, cellulosic/fibrous or other solid materials. There also may be one or more purification steps to remove, for example, certain constituents and/or contaminants. Such purification may be accomplished, for example, by distillation, evaporation, centrifugation, etc. There also may be one or more concentration and/or drying steps to remove water and/or other volatiles, e.g. alcohol, lighter compounds, VOCs, etc. Moreover, acids and/or bases may be added to adjust pH or neutralize. Depending on the desired form of the final/end botanical extract, one can also utilize various additional steps understood in the art, such as screening, pressing, milling, grinding, mixing, dispersing, etc. It is to be appreciated that combinations of these additional processing steps in duplicative and/or different orders is also contemplated.

### Potentilla Glabra

In some embodiments, the botanical active component, and thus the composition, comprises the extract of Potentilla glabra, i.e., an extract comprising, optionally consisting essentially of material from the flowering plant species Potentilla glabra, such as the leaf. The Potentilla glabra extract is not particularly limited, and may comprise or be any leaf extract or combination of extracts from a Potentilla glabra plant suitable for use in the embodiments herein. More specifically, exemplary Potentilla glabra leaf extracts include those capable of inhibiting elastase activity or eliciting/exhibiting any other such activities described herein as part of the botanical active component.

Potentilla glabra has been reported to contain various bioactive components, such as phenolic acids, flavonoids, terpenoids, triterpenes, tannins, polyphenols, saponins, polysaccharides, and other compounds. Potentilla glabra may simply be referred to as P. glabra, or via various other names such as Potentilla glabra Lodd., Potentilla glabra G. Lodd., Dasiphora glabra (G. Lodd.) Soják, Silver Dew Plum, Yinlumei, or [symbol] . The leaves and flowers of Potentilla glabra Lodd. may be referred to as Yaowang tea. In various embodiments, Potentilla glabra may be of the variety Potentilla glabra Lodd. var. mandshurica (Maxim.) Hand.-Mazz, which may be referred to as Baimao Yinlumei, [symbol] , or [symbol] [symbol] ).

Specific examples of Potentilla glabra extracts are known in art. As such, the Potentilla glabra extract may be purchased or otherwise obtained commercially from various sources, prepared (e.g. using any conventional extraction technique(s) known in the art, such as any of those described herein), or combinations thereof. In certain embodiments, the extract of Potentilla glabra is obtained by alcohol extracting (e.g., ethanol extracting) plant material of Potentilla glabra, including but not limited to the leaf. In other embodiments, the extract of Potentilla glabra may be obtained by water extracting (or aqueous extracting) plant material of Potentilla glabra.

In particular embodiments, the extract of Potentilla glabra leaf is obtained by alcohol extracting (e.g., ethanol extracting) leaves or leaf-based plant material of Potentilla glabra. The leaves may be fresh or dried, typically dried to prevent decay. The dried leaves may then be formed into a powder, which can be used as the extract itself, or more typically, the powdered leaf is further processed to form the extract as like described below.

As will be understood by those of skill in the art, Potentilla glabra is primarily cultivated for its leaf. As such, in various embodiments, the extract of Potentilla glabra is an extract of Potentilla glabra leaf. Suitable extractions include those noted above, e.g., ethanol and water extractions of leaves. The leaves can be from one or more plants, and can be fresh, dried, or otherwise aged.

Byway of example, certain extracts can be obtained where Potentilla glabra (e.g., leaf) is pulverized to a homogeneous size in a mill. Next, the resulting powder is extracted using an ethanol solution. The solution is then filtered and the filtrate can be concentrated under reduced pressure to yield a syrup. The syrup can then be freeze-dried to dryness to obtain extract.

In various embodiments, the botanical active component consists of the extract of Potentilla glabra leaf. In further or other embodiments, the composition is substantially to completely free of components obtained from non-leaf-based plant material of Potentilla glabra. In these embodiments, the non-leaf-based plant material of Potentilla glabra can be, for example, the root, stem, bark, rhizome, seed, or flower of a Potentilla glabra plant. Without being bound by any particular theory, it is believed that the leaf of Potentilla glabra is most useful for elastase inhibition effect; whereas other parts of Potentilla glabra are not (as illustrated in the Examples section below).

Suitable leaf extracts may be processed (e.g. defatted, partially defatted, ground, dried, precipitated, washed, filtered, mesh-sorted, extracted, distilled, concentrated, etc.) to obtain the Potentilla glabra leaf extract. Likewise, Potentilla glabra leaf may be extracted in raw form, or processed prior to extraction of the Potentilla glabra leaf extract (e.g. used in raw form, suspended form, dehydrated form, concentrated form, etc.). In other embodiments, the Potentilla glabra extract may comprise material from any part of the plant, or combinations of parts, and is not limited to leaf extract. For example, the Potentilla glabra extract may comprise material extracted from one or more parts of a Potentilla glabra plant, including the root, stem, bark, rhizome, leaf, bud, flower, seed, and/or fruit, thereof.

The amount of the Potentilla glabra extract utilized in the botanical active component may vary, and will be selected based on the number and types of components being utilized in the botanical active component. In certain embodiments, the botanical active component comprises from 1 to 2000 mg of the Potentilla glabra extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Potentilla glabra extract in an amount of at least 1, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Potentilla glabra extract in an amount of ≤100, ≤250, ≤500, ≤750, ≤1000, ≤2000, ≤5000 mg. In various embodiments, the botanical active component can include an amount of Potentilla glabra extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, 540, ≤50, ≤60, ≤70, ≤80, ≤90, and ≤99 wt. %, respectively, based on the total weight of the botanical active component.

In certain embodiments, the botanical active component comprises more than one Potentilla glabra extract, such as 2, 3, 4, or more Potentilla glabra extracts. In such embodiments, each Potentilla glabra extract is independently selected, may be the same as or different from any other Potentilla glabra extract, and each utilized in an amount as described above.

The Potentilla glabra extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Potentilla glabra extract(s) utilized. It will be appreciated that the Potentilla glabra extract may be combined with the carrier vehicle, if utilized, prior to, during, or after being combined with any other components of the botanical active component and/or composition.

Bombax malabaricum/Bombax ceiba

In some embodiments, the botanical active component, and thus the composition, comprises the extract of Bombax malabaricum, i.e., an extract comprising, optionally consisting essentially of material from the plant species Bombax malabaricum, such as the flower. The Bombax malabaricum extract is not particularly limited, and may comprise or be any flower extract or combination of extracts from a Bombax malabaricum plant suitable for use in the embodiments herein. More specifically, exemplary Bombax malabaricum flower extracts include those capable of inhibiting elastase activity or eliciting/exhibiting any other such activities described herein as part of the botanical active component.

Bombax malabaricum, also known as Bombax ceiba, has been reported to contain various bioactive components, such as flavonoids, flavanols, terpenoid saponins, tannins, sterols, phenols, pyranosides, polysaccharides, glucosides, sesquiterpenoids, steroids, napthequionones, neolignans, alkanes, fatty acids, monoterpenes, organic acids and other compounds. Bombax malabaricum may simply be referred to as B. malabaricum, or via various other names such as Bombax malabaricum DC., Bombax ceiba Linnaeus, Bombax ceiba Linn., Bombax ceiba L., B. ceiba, Gossampinus Malabarica, Panzhihua, Kapok Tree, Hero Tree, Banzhi, Qiongzhi, Malabar silk-cotton tree, red silk-cotton, red cotton tree, silk-cotton, Common Bombax Flower, Mu Mian Hua, [symbol] , or [symbol] .

Specific examples of Bombax malabaricum extracts are known in art. As such, the Bombax malabaricum extract may be purchased or otherwise obtained commercially from various sources, prepared (e.g. using any conventional extraction technique(s) known in the art, such as any of those described herein), or combinations thereof. In certain embodiments, the extract of Bombax malabaricum is obtained by alcohol extracting (e.g., ethanol extracting) plant material of Bombax malabaricum, including but not limited to the flower. In other embodiments, the extract of Bombax malabaricum may be obtained by water extracting (or aqueous extracting) plant material of Bombax malabaricum.

As will be understood by those of skill in the art, Bombax malabaricum is primarily cultivated for its flower, leaves, and/or fruit. As such, in various embodiments, the extract of Bombax malabaricum is an extract of Bombax malabaricum flower. Suitable extractions include those noted above, e.g., ethanol and water extractions of flowers. In particular embodiments, the extract of Bombax malabaricum flower is obtained by alcohol extracting (e.g., ethanol extracting) flower or flower-based plant material of Bombax malabaricum. The flowers can be from one or more plants, and can be fresh, dried, or otherwise aged.

By way of example, certain extracts can be obtained where Bombax malabaricum (e.g. flower) is pulverized to a homogeneous size in a mill. Next, the resulting powder is extracted using an ethanol solution. The solution is then filtered and the filtrate can be concentrated under reduced pressure to yield a syrup. The syrup can then be freeze-dried to dryness to obtain extract.

In various embodiments, the botanical active component consists of the extract of Bombax malabaricum flower. In further or other embodiments, the composition is substantially to completely free of components obtained from non-flower-based plant material of Bombax malabaricum. In these embodiments, the non-flower-based plant material of Bombax malabaricum can be, for example, the root, stem, bark, rhizome, leaf, seed, or fruit of a Bombax malabaricum plant. Without being bound by any particular theory, it is believed that the flower of Bombax malabaricum is most useful for elastase inhibition effect; whereas other parts of Bombax malabaricum are not (as illustrated in the Examples section below).

Suitable flower extracts may be processed (e.g. defatted, partially defatted, ground, dried, precipitated, washed, filtered, mesh-sorted, extracted, distilled, concentrated, etc.) to obtain the Bombax malabaricum flower extract. Likewise, Bombax malabaricum flower may be extracted in raw form, or processed prior to extraction of the Bombax malabaricum flower extract (e.g. used in raw form, suspended form, dehydrated form, concentrated form, etc.). In other embodiments, the Bombax malabaricum extract may comprise material from any part of the plant, or combinations of parts, and is not limited to flower extract. For example, the Bombax malabaricum extract may comprise material extracted from one or more parts of a Bombax malabaricum plant, including the root, stem, bark, rhizome, leaf, bud, flower, seed, and/or fruit, thereof.

The amount of the Bombax malabaricum extract utilized in the botanical active component may vary, and will be selected based on the number and types of components being utilized in the botanical active component. In certain embodiments, the botanical active component comprises from 1 to 2000 mg of the Bombax malabaricum extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Bombax malabaricum extract in an amount of at least 1, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Bombax malabaricum extract in an amount of ≤100, ≤250, ≤500, ≤750, ≤1000, ≤2000, ≤5000 mg. In various embodiments, the botanical active component can include an amount of Bombax malabaricum extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, ≤40, ≤50, ≤60, ≤70, ≤80, ≤90, and ≤99 wt. %, respectively, based on the total weight of the botanical active component.

In certain embodiments, the botanical active component comprises more than one Bombax malabaricum extract, such as 2, 3, 4, or more Bombax malabaricum extracts. In such embodiments, each Bombax malabaricum extract is independently selected, may be the same as or different from any other Bombax malabaricum extract, and each utilized in an amount as described above.

The Bombax malabaricum extract maybe utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Bombax malabaricum extract(s) utilized. It will be appreciated that the Bombax malabaricum extract may be combined with the carrier vehicle, if utilized, prior to, during, or after being combined with any other components of the botanical active component and/or composition.

Amomum villosum

In some embodiments, the botanical active component, and thus the composition, comprises the extract of Amomum villosum, i.e., an extract comprising, optionally consisting essentially of material from the plant species Amomum villosum, such as the seed. The Amomum villosum extract is not particularly limited, and may comprise or be any seed extract or combination of extracts from a Amomum villosum plant suitable for use in the embodiments herein. More specifically, exemplary Amomum villosum seed extracts include those capable of inhibiting elastase activity or eliciting/exhibiting any other such activities described herein as part of the botanical active component.

Amomum villosum has been reported to contain various bioactive components, such as terpenoids, flavonoids, diarylheptanoids, and coumarins, including but not limited to delta-camphor, delta-borneol, delta-bornyl acetate, delta-limonene, alpha-pinene, phellandrene, paramethoxyethyl cinnamate, nerolidol, linalool, liquiritin, and glucovanillic acid, and other compounds. Amomum villosum may simply be referred to as A. villosum, or via various other names such as Amomum villosum Lour, Wurbainia villosa, Malabar cardamom, Tavoy cardamom, false cardamom, Sha Ren, or [symbol] ).

Specific examples of Amomum villosum extracts are known in art. As such, the Amomum villosum extract may be purchased or otherwise obtained commercially from various sources, prepared (e.g. using any conventional extraction technique(s) known in the art, such as any of those described herein), or combinations thereof. In certain embodiments, the extract of Amomum villosum is obtained by alcohol extracting (e.g., ethanol extracting) plant material of Amomum villosum, including but not limited to the seed. In other embodiments, the extract of Amomum villosum may be obtained by water extracting (or aqueous extracting) plant material of Amomum villosum.

As will be understood by those of skill in the art, Amomum villosum is primarily cultivated for its fruit and accompanying seeds. As such, in various embodiments, the extract of Amomum villosum is an extract of Amomum villosum seed. Suitable extractions include those noted above, e.g., ethanol and water extractions of seeds. In particular embodiments, the extract of Amomum villosum seed is obtained by alcohol extracting (e.g., ethanol extracting) seed or seed-based plant material of Amomum villosum. The seeds can be from one or more plants, and can be fresh, dried, or otherwise aged.

By way of example, certain extracts can be obtained where Amomum villosum (e.g. seed) is pulverized to a homogeneous size in a mill. Next, the resulting powder is extracted using an ethanol solution. The solution is then filtered and the filtrate can be concentrated under reduced pressure to yield a syrup. The syrup can then be freeze-dried to dryness to obtain extract.

In various embodiments, the botanical active component consists of the extract of Amomum villosum seed. In further or other embodiments, the composition is substantially to completely free of components obtained from non-seed-based plant material of Amomum villosum. In these embodiments, the non-seed-based plant material of Amomum villosum can be, for example, the root, stem, bark, rhizome, leaf, flower, or fruit of an Amomum villosum plant. Without being bound by any particular theory, it is believed that the seed of Amomum villosum is most useful for elastase inhibition effect; whereas other parts of Amomum villosum are not (as illustrated in the Examples section below).

Suitable seed extracts may be processed (e.g. defatted, partially defatted, ground, dried, precipitated, washed, filtered, mesh-sorted, extracted, distilled, concentrated, etc.) to obtain the Amomum villosum seed extract. Likewise, Amomum villosum seed may be extracted in raw form, or processed prior to extraction of the Amomum villosum seed extract (e.g. used in raw form, suspended form, dehydrated form, concentrated form, etc.). In other embodiments, the Amomum villosum extract may comprise material from any part of the plant, or combinations of parts, and is not limited to seed extract. For example, the Amomum villosum extract may comprise material extracted from one or more parts of a Amomum villosum plant, including the root, stem, bark, rhizome, leaf, bud, flower, seed, and/or fruit, thereof.

The amount of the Amomum villosum extract utilized in the botanical active component may vary, and will be selected based on the number and types of components being utilized in the botanical active component. In certain embodiments, the botanical active component comprises from 1 to 2000 mg of the Amomum villosum extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Amomum villosum extract in an amount of at least 1, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Amomum villosum extract in an amount of ≤100, ≤250, ≤500, ≤750, ≤1000, ≤2000, ≤5000 mg. In various embodiments, the botanical active component can include an amount of Amomum villosum extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, 540, ≤50, ≤60, ≤70, ≤80, ≤90, and ≤99 wt. %, respectively, based on the total weight of the botanical active component.

In certain embodiments, the botanical active component comprises more than one Amomum villosum extract, such as 2, 3, 4, or more Amomum villosum extracts. In such embodiments, each Amomum villosum extract is independently selected, may be the same as or different from any other Amomum villosum extract, and each utilized in an amount as described above.

The Amomum villosum extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Amomum villosum extract(s) utilized. It will be appreciated that the Amomum villosum extract may be combined with the carrier vehicle, if utilized, prior to, during, or after being combined with any other components of the botanical active component and/or composition.

Amomum kravanh

In some embodiments, the botanical active component, and thus the composition, comprises the extract of Amomum kravanh, i.e., an extract comprising, optionally consisting essentially of material from the plant species Amomum kravanh, such as the fruit. The Amomum kravanh extract is not particularly limited, and may comprise or be any fruit extract or combination of extracts from a Amomum kravanh plant suitable for use in the embodiments herein. More specifically, exemplary Amomum kravanh fruit extracts include those capable of inhibiting elastase activity or eliciting/exhibiting any other such activities described herein as part of the botanical active component.

Amomum kravanh has been reported to contain various bioactive components, such as flavonoids, monoterpenoids, monoterpenes, tetracyclic diterpenes, diarylheptanoids, steroids, coumarins, and secolignan, including but not limited to 1,8-cineole, bornyl acetate, α-pinene, p-pinene, α-terpinene, terpineol, linalool, and limonene, and other compounds. Amomum kravanh may simply be referred to as A. kravanh, or via various other names such as Amomum kravanh Pierre ex Gagnep, Amomum Cardamomum, Round Cardamom, Cambodian cardamom, white cardamom, Bai Dou Kou, or [symbol] .

Specific examples of Amomum kravanh extracts are known in art. As such, the Amomum kravanh extract may be purchased or otherwise obtained commercially from various sources, prepared (e.g. using any conventional extraction technique(s) known in the art, such as any of those described herein), or combinations thereof. In certain embodiments, the extract of Amomum kravanh is obtained by alcohol extracting (e.g., ethanol extracting) plant material of Amomum kravanh, including but not limited to the fruit. In other embodiments, the extract of Amomum kravanh may be obtained by water extracting (or aqueous extracting) plant material of Amomum kravanh including but not limited to the fruit.

As will be understood by those of skill in the art, Amomum kravanh is primarily cultivated for its fruit and accompanying seeds. As such, in various embodiments, the extract of Amomum kravanh is an extract of Amomum kravanh fruit. Suitable extractions include those noted above, e.g., ethanol and water extractions of fruits. In particular embodiments, the extract of Amomum kravanh fruit is obtained by alcohol extracting (e.g., ethanol extracting) fruit or fruit-based plant material of Amomum kravanh. The fruits can be from one or more plants, and can be fresh, dried, or otherwise aged.

By way of example, certain extracts can be obtained where Amomum kravanh (e.g. fruit) is pulverized to a homogeneous size in a mill. Next, the resulting powder is extracted using an ethanol solution. The solution is then filtered and the filtrate can be concentrated under reduced pressure to yield a syrup. The syrup can then be freeze-dried to dryness to obtain extract.

In various embodiments, the botanical active component consists of the extract of Amomum kravanh fruits. In further or other embodiments, the composition is substantially to completely free of components obtained from non-fruit-based plant material of Amomum kravanh. In these embodiments, the non-fruit-based plant material of Amomum kravanh can be, for example, the root, stem, bark, rhizome, leaf, flower of an Amomum kravanh plant. Without being bound by any particular theory, it is believed that the fruits of Amomum kravanh is most useful for elastase inhibition effect; whereas other parts of Amomum kravanh are not (as illustrated in the Examples section below).

Suitable fruit extracts maybe processed (e.g. defatted, partially defatted, ground, dried, precipitated, washed, filtered, mesh-sorted, extracted, distilled, concentrated, etc.) to obtain the Amomum kravanh fruit extract. Likewise, Amomum kravanh fruit may be extracted in raw form, or processed prior to extraction of the Amomum kravanh fruit extract (e.g. used in raw form, suspended form, dehydrated form, concentrated form, etc.). In other embodiments, the Amomum kravanh extract may comprise material from any part of the plant, or combinations of parts, and is not limited to fruit extract. For example, the Amomum kravanh extract may comprise material extracted from one or more parts of a Amomum kravanh plant, including the root, stem, bark, rhizome, leaf, bud, flower, seed, and/or fruit, thereof.

The amount of the Amomum kravanh extract utilized in the botanical active component may vary, and will be selected based on the number and types of components being utilized in the botanical active component. In certain embodiments, the botanical active component comprises from 1 to 2000 mg of the Amomum kravanh extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Amomum kravanh extract in an amount of at least 1, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Amomum kravanh extract in an amount of ≤100, ≤250, ≤500, ≤750, ≤1000, ≤2000, ≤5000 mg. In various embodiments, the botanical active component can include an amount of Amomum kravanh extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary may be selected to be generally ≤10, ≤20, ≤30, 540, ≤50, ≤60, ≤70, ≤80, ≤90, and ≤99 wt. %, respectively, based on the total weight of the botanical active component.

In certain embodiments, the botanical active component comprises more than one Amomum kravanh extract, such as 2, 3, 4, or more Amomum kravanh extracts. In such embodiments, each Amomum kravanh extract is independently selected, may be the same as or different from any other Amomum kravanh extract, and each utilized in an amount as described above.

The Amomum kravanh extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Amomum kravanh extract(s) utilized. It will be appreciated that the Amomum kravanh extract may be combined with the carrier vehicle, if utilized, prior to, during, or after being combined with any other components of the botanical active component and/or composition.

Cornus officinalis

In some embodiments, the botanical active component, and thus the composition, comprises the extract of Cornus officinalis, i.e., an extract comprising, optionally consisting essentially of material from the plant species Cornus officinalis, such as the fruit. The Cornus officinalis extract is not particularly limited, and may comprise or be any fruit extract or combination of extracts from a Cornus officinalis plant suitable for use in the embodiments herein. More specifically, exemplary Cornus officinalis fruit extracts include those capable of inhibiting elastase activity or eliciting/exhibiting any other such activities described herein as part of the botanical active component.

Cornus officinalis has been reported to contain various bioactive components, such as iridoids, glucosides, iridoid glucosides, secoiridoid glycosides, loganins, cornusides, cornusphenosides, triterpenoids, gallotannins, 2-furancarboxaldehyde, and other compounds.

Cornus officinalis may simply be referred to as C. officinalis, or via various other names such as Cornus officinalis Sieb. et Zucc., Macrocarpium Officinale, Japanese Cornelian Cherry, Japanese Cornelian Dogwood, Asiatic Dogwood, Asiatic Cornelian Cherry, Shan Zhu Yu, or [symbol] [symbol] .

Specific examples of Cornus officinalis extracts are known in art. As such, the Cornus officinalis extract may be purchased or otherwise obtained commercially from various sources, prepared (e.g. using any conventional extraction technique(s) known in the art, such as any of those described herein), or combinations thereof. In certain embodiments, the extract of Cornus officinalis is obtained by alcohol extracting (e.g., ethanol extracting) plant material of Cornus officinalis, including but not limited to the fruit. In other embodiments, the extract of Cornus officinalis may be obtained by water extracting (or aqueous extracting) plant material of Cornus officinalis including but not limited to the fruit.

As will be understood by those of skill in the art, Cornus officinalis is primarily cultivated for its fruit. As such, in various embodiments, the extract of Cornus officinalis is an extract of Cornus officinalis fruit. Suitable extractions include those noted above, e.g., ethanol and water extractions of fruits. In particular embodiments, the extract of Cornus officinalis fruit is obtained by alcohol extracting (e.g., ethanol extracting) fruit or fruit-based plant material of Cornus officinalis. The fruits can be from one or more plants, and can be fresh, dried, or otherwise aged.

Byway of example, certain extracts can be obtained where Cornus officinalis (e.g. fruit) is pulverized to a homogeneous size in a mill. Next, the resulting powder is extracted using an aqueous or ethanol solution. The solution is then filtered and the filtrate can be concentrated under reduced pressure to yield a syrup. The syrup can then be freeze-dried to dryness to obtain extract.

In various embodiments, the botanical active component consists of the extract of Cornus officinalis fruit. In further or other embodiments, the composition is substantially to completely free of components obtained from non-fruit-based plant material of Cornus officinalis. In these embodiments, the non-fruit-based plant material of Cornus officinalis can be, for example, the root, stem, bark, rhizome, leaf, flower, or seed of an Cornus officinalis plant. Without being bound by any particular theory, it is believed that the fruit of Cornus officinalis is most useful for elastase inhibition effect; whereas other parts of Cornus officinalis are not (as illustrated in the Examples section below).

Suitable fruit extracts maybe processed (e.g. defatted, partially defatted, ground, dried, precipitated, washed, filtered, mesh-sorted, extracted, distilled, concentrated, etc.) to obtain the Cornus officinalis fruit extract. Likewise, Cornus officinalis fruit may be extracted in raw form, or processed prior to extraction of the Cornus officinalis fruit extract (e.g. used in raw form, suspended form, dehydrated form, concentrated form, etc.). In other embodiments, the Cornus officinalis extract may comprise material from any part of the plant, or combinations of parts, and is not limited to fruit extract. For example, the Cornus officinalis extract may comprise material extracted from one or more parts of a Cornus officinalis plant, including the root, stem, bark, rhizome, leaf, bud, flower, seed, and/or fruit, thereof.

The amount of the Cornus officinalis extract utilized in the botanical active component may vary, and will be selected based on the number and types of components being utilized in the botanical active component. In certain embodiments, the botanical active component comprises from 1 to 2000 mg of the Cornus officinalis extract, such as from 1 to 1000, optionally of from 2 to 800, optionally of from 20 to 750, or optionally of from 50 to 500, mg. However, amounts outside these ranges may also be utilized. For example, in certain embodiments, the botanical active component includes the Cornus officinalis extract in an amount of at least 1, optionally of at least about 20, optionally of at least about 50, optionally of at least 100, optionally of at least 250, optionally of at least 500, optionally of at least 1000, or optionally of at least 1500, mg. In these or other embodiments, an upper boundary may be selected such that the botanical active component comprises the Cornus officinalis extract in an amount of ≤100, ≤250, ≤500, ≤750, ≤1000, ≤2000, ≤5000 mg. In various embodiments, the botanical active component can include an amount of Cornus officinalis extract optionally in an amount of greater than 1, optionally greater than 5, optionally greater than 10, optionally greater than 25, optionally greater than 50, optionally greater than 75, optionally greater than 80, or optionally greater than 95, wt. %, based on the total weight of the botanical active component. In such embodiments, an upper boundary maybe selected to be generally ≤10, ≤20, ≤30, 540, ≤50, ≤60, ≤70, ≤80, ≤90, and ≤99 wt. %, respectively, based on the total weight of the botanical active component.

In certain embodiments, the botanical active component comprises more than one Cornus officinalis extract, such as 2, 3, 4, or more Cornus officinalis extracts. In such embodiments, each Cornus officinalis extract is independently selected, may be the same as or different from any other Cornus officinalis extract, and each utilized in an amount as described above.

The Cornus officinalis extract may be utilized in any form, such as neat (i.e., absent solvents, carrier vehicles, diluents, etc.), or disposed in a carrier vehicle, such as a solvent or dispersant. The carrier vehicle, if present, may comprise an aqueous solvent (e.g. water), an organic solvent, fluid, or oil, or the like, or combinations thereof. When utilized, the carrier vehicle will be selected based on the particular components of the botanical active component and/or the composition, such as the particular Cornus officinalis extract(s) utilized. It will be appreciated that the Cornus officinalis extract may be combined with the carrier vehicle, if utilized, prior to, during, or after being combined with any other components of the botanical active component and/or composition.

### Definitions

In order to provide a clear and consistent understanding of the specification and claims, the following definitions are provided.

“Improving at least one sign of aging” and “improving a sign of aging” are used interchangeably herein to designate preventing, arresting, reversing, ameliorating, diminishing, and/or reducing a sign of aging. Representative signs of aging include, but are not limited to, lines, fine lines, wrinkles, crow's feet, dark eye circles, blemishes, age spots, stretch marks, or combinations thereof.

“Improving the appearance of skin” and “improving the aesthetic appearance of skin” are used interchangeably herein to designate an aesthetic improvement in the appearance of skin. Representative improvements may include, but are not limited to, favorable characteristics and/or properties related skin thickness, elasticity, resiliency, moisturization, smoothness, tone, texture, radiance, luster, brightness, clarity, contour, firmness, tautness, suppleness, softness, sensitivity, pore size, or combinations thereof. These terms may also be used to designate an improvement in an adverse skin condition. Representative adverse conditions affecting by, resulting in or resulting from such an adverse skin condition include, but are not limited to, psoriasis, eczema, seborrhea, dermatitis, sunburn, estrogen imbalance, hyperpigmentation, hypopigmentation, discoloration, yellowing, freckles, skin atrophy, skin breakout, skin fragility, dryness, tactile roughness, chapping, sagginess, thinning, hyperplasia, fibrosis, enlarged pores, cellulite formation, bruising, acne formation, apoptosis, cellular differentiation, cellular de-differentiation, prevention of tumor induction or tumor progression, viral infections, fungal infections, bacterial infections, spider veins (telangectasia), hirsutism, rosacea, pruritis, calluses, warts, corns, or combinations thereof.

The terms “composition” or “formulation” refer to a product that treats, improves, promotes, increases, manages, controls, maintains, optimizes, modifies, reduces, inhibits, or prevents a particular condition associated with a natural state, biological process or disease or disorder. For example, a composition or a formulation improves at least one sign of aging skin in a subject and/or minimizes or inhibits glycation in skin (e.g., mature skin). The terms composition and formulation include, but are not limited to, pharmaceutical (i.e., drug), over-the counter (OTC), cosmetic, food, food ingredient or dietary supplement compositions that include an effective amount of an extract, at least one component thereof, or a mixture thereof.

Exemplary compositions and/or formulations include cream, cosmetic lotion, pack or powder, or as an emulsion, lotion, liniment foam, tablets, plasters, granules, or ointment. Preferred compositions are formulated for topical application/administration and for oral administration/ingestion.

As used herein, the term “effective amount” or “therapeutically effective amount” of a pure compound, composition, extract, extract mixture, component of the extract, and/or active agent or ingredient, or a combination thereof refers to an amount effective at dosages and for periods of time sufficient to achieve a desired result. For example, the “effective amount” or “therapeutically effective amount” refers to that amount of a pure compound, composition, extract, botanical extract, extract mixture, botanical extract mixture, component of the extract, and/or active agent or ingredient, or a combination thereof of this invention which, when administered to a subject (e.g., mammal, such as a human), is sufficient to effect treatment, such as improving aging skin and/or minimizing or inhibiting glycation in mature skin. The amount of a composition, extract, botanical extract, extract mixture, botanical extract mixture, component of the extract, and/or active agent or ingredient of this disclosure that constitutes an “effective amount” or “therapeutically effective treatment” will vary depending on the active agent or the compound, the condition being treated and its severity, the manner of administration, the duration of treatment, or the age of the subject to be treated, but can be determined routinely by one of ordinary skill in the art having regard to his own knowledge and to this disclosure.

The term “pharmaceutically acceptable” means those drugs, medicaments, extracts or inert ingredients, which are suitable for use in contact with the tissues of humans and lower animals without undue toxicity, incompatibility, instability, irritation, and the like, commensurate with a reasonable benefit/risk ratio.

The terms “applying” and “administering” are defined as providing a composition to a subject via a route known in the art, including but not limited to topical, intravenous, intra-arterial, oral, parenteral, buccal, transdermal, rectal, intramuscular, subcutaneous, intraosseous, transmucosal, or intraperitoneal routes of administration. In preferred embodiments, topical and/or oral routes of administering the described composition are suitable.

The terms “minimize,” “reduce,” “suppress,” “decrease” and/or “inhibit” refer to a decrease or reduction in elastase activity and/or expression, and/or its downstream effect, in the presence of a botanical (or plant) ingredient or botanical extract as described herein, when compared to elastase activity and/or expression in the absence of a botanical ingredient or botanical extract as described herein, such as in a control sample. The degree of decrease or inhibition of elastase activity and/or expression, and/or its downstream effect, will vary with the nature and quantity of a botanical ingredient or botanical extract present, but will be evident, e.g., as a detectable decrease in elastase activity and/or expression; desirably a degree of decrease greater than about 5%, about 10%, about 15%, about 20%, about 25%, about 50%, about 75%, about 90%, about 95% or about 99% (or any degree of decrease in the range of from about 5% to about 99%) as compared to elastase activity and/or expression in the absence of the botanical ingredient or botanical extract. For example, a composition comprising a plant ingredient or plant extract of one or more of Potentilla glabra, Bombax malabaricum, Amomum villosum, Amomum kravanh, and/or Cornus officinalis can minimize or reduce elastase activity in skin, such as a mature skin.

As used herein, the term “subject” or “individual” includes mammals to which a composition may be administered. Non-limiting examples of mammals include humans, non-human primates, rodents (including transgenic and non-transgenic mice) or the like. In some embodiments, the subject is a mammal, and in some embodiments, the subject is human.

### Compositions (or Formulations)

The composition may include any amount of the botanical active component, which will be selected based on the number and types of components being utilized in the composition as a whole. In general, the botanical active component is present in the composition in an amount effective to inhibit elastase in the skin of the subject.

In certain embodiments, composition comprises the botanical active component in an amount of from 1 to 5000, optionally of from 2 to 2000, optionally of from 5 to 1750, optionally of from 10 to 1500, optionally of from 15 to 1250, optionally of from 20 to 1000, optionally of from 25 to 750, optionally of from 30 to 500, optionally of from 35 to 500, optionally of from 40 to 500, optionally of from 45 to 450, optionally of from 50 to 450, or optionally of from 50 to 400, mg. However, amounts outside and/or overlapping with these ranges may also be utilized. For example, it is to be appreciated that the ranges described above with respect to the amount of each botanical extract in the botanical active component may equally apply to the amount of each botanical extract in the composition as a whole, such as when the botanical active component consists of but one of the botanical extracts.

In specific embodiments of the composition, the formulation comprises: one or more of an ethanol extract of the leaf of Potentilla glabra Lodd. var. mandshurica (Maxim.) Hand.-Mazz., an ethanol extract of the flower of Bombax malabaricum DC, an ethanol extract of the seed of Amomum villosum Lour, an ethanol extract of the fruit of Amomum kravanh Pierre ex Gagnep, and an aqueous and/or ethanol extract of the fruit of Cornus officinalis Sieb. et Zucc.

A flavoring essence and/or sugar substitute may be included in the composition and can be any type of conventional component, e.g., flavoring agents, understood in the art. The plant (or botanical) extracts can each be as described above. Examples of suitable flavoring agents are described further below.

In general, the composition is not limited in terms of formulation, peripheral ingredients, form, number of functions, etc., aside from comprising the botanical active component and the botanical extract(s) thereof. Rather, the composition may be varied, and may be formulated in any fashion consistent with this disclosure.

Typically, the composition is formulated or otherwise adapted for administration to a mammalian subject (e.g. a human). For example, in various embodiments, the composition is adapted to be topically administrated (e.g. eye creams, masks) or consumed and/or orally administered to a human subject.

In certain embodiments, the composition is further defined as a topical composition that is formulated for topical administration to the subject. In such embodiments, the composition may also be referred to as a cosmetic composition, and typically comprises at least one cosmetically acceptable carrier in addition to the bioactive agent composition. In specific embodiments, the cosmetically acceptable carrier is not naturally occurring. In other words, the carrier is not a product of nature in these specific embodiments. In other embodiments, the carrier is selected from conventional carriers understood in the art, and can be used in conventional amounts.

In other certain embodiments, the composition is further defined as an oral composition that is formulated for oral administration to the subject. In such embodiments, the composition may also be referred to as an ingestible composition, and typically comprises at least one pharmaceutically acceptable additive in addition to the bioactive agent composition. In specific embodiments, the pharmaceutically acceptable additive is not naturally occurring. In other words, the pharmaceutically acceptable additive is not a product of nature in these specific embodiments. In other embodiments, the pharmaceutically acceptable additive is selected from conventional additives understood in the art, and can be used in conventional amounts.

As such, it should be appreciated that the particular additives, carriers, adjuvants, fillers, etc. present in or combined with the composition may vary. Moreover, the physical form of the composition is not limited, and will be selected based on the particular components of the composition, a desired use of the composition, etc. As such, as will be understood in view of the description herein, the composition may be formulated as a liquid, dry powder, suspension, emulsion, gel, paste, etc., and combinations thereof. In certain embodiments, the composition is formulated as a sterile, non-pyrogenic liquid solution or suspension, a coated capsule, a suppository, a lyophilized powder, a transdermal patch, a softgel, or other forms are known.

Other examples of suitable forms include solids, gels, liquids, creams, lotions, pomades, mousses, powders, foams, sprays, ointments, or other such preparations where the botanical active component is disposed in an appropriate carrier vehicle, such as any of those described herein. In particular embodiments, the composition is formulated or otherwise provided as an eye cream or mask for topical application.

The composition can be prepared using various methods. For example, actives of the composition (such as the botanical extract(s)), and optionally one or more inactives (such as one or more conventional components, additives, excipients, etc.), can be mixed or blended and compressed or compounded utilizing various techniques understood in the art. The composition of this disclosure is not limited to a particular order of manufacturing steps or method of manufacture.

In various embodiments, the composition is administered topically by application to the subject's skin. The subject is typically a human, and can include men and women of various ages. The method/composition of this disclosure is not limited to a particular subject.

The composition can be in various forms. Examples of suitable forms include solids, gels and liquids. For example, the composition can be formulated for application as a gel, cream, lotion, pomade, mousse, powder, or foam for application to the subject's skin. In another example, the composition can be formulated for spraying onto a subject's skin. The composition can be formulated to be sprayed as either an aerosol spray or pump spray. In still another example, the composition can be formulated for application using a pre-moistened towelette. In another example, the composition can be formulated as a solid that is rubbed onto the subject's skin. In another example, the composition is formulated for delivery through a patch that is adhered to the subject's skin.

Other than the botanical active component (i.e., the “actives” or “active ingredients”), the composition can include pharmaceutically acceptable additives that are inactives (or “inactive ingredients”) including, but not limited to, excipients, such as diluents and binders; granulating agents; glidants (or flow aids); fillers; lubricants; preservatives; stabilizers; coatings; disintegrants; fragrances; and pigments. The active ingredients and the pharmaceutically acceptable additives can be combined or compounded as desired to form an individual dose that provides the desired amount of active ingredient to the human subject when topically applied.

Optionally, the composition may include one or more additional components such as additives. Suitable additives include those understood in the art, including but not limited to, moisturizers, emollients, emulsifiers, surfactants, oils, extracts, skin protectants, disinfectants, antiseptics, drugs and drug substances, analgesic compounds, anti-neuralgic compound, anti-oxidants, blood circulation promoters, antidepressant compounds, anti-anxiety compounds, anti-stress compounds, sunscreens, insect repellants, preservatives, exfoliants, fragrances, colors, fillers, solvents, vehicles, carriers, other types of additives known to those of skill in the art, and combinations thereof. Such additives may be utilized alone or in combination. In general, the optional additives may be of any type used in personal care products and cosmetic products.

Excipients can be further classified as other components. Specifically, excipients used in oral solid dosage forms have been classified based on their functionality into groups such as diluents, disintegrants, binders, compression aids, granulating agents, glidants, lubricants, release-controlling polymers, stabilizers (such as antioxidants, chelators, and pH-modifiers), film-coating polymers, coating agents, vehicles, plasticizers, surfactants, colorants, sweeteners, and flavors.

In various embodiments, the composition comprises at least one component selected from the group consisting of binders, lubricants, glidants, and combinations thereof. In certain embodiments, the composition includes one or more compounds including, but not limited to, methylcellulose, hydroxypropyl methylcellulose, ethyl cellulose, cellulose acetate phthalate, acacia, gums, wax, glycerol monostearate, acrylic acid polymers and copolymers, methacrylic acid, methyl acrylate, ethyl acrylate, methyl methacrylate, ethyl methacrylate, lactose, calcium sulfate, calcium phosphate dibasic, sugar, microcrystalline cellulose (MCC), starch, sodium starch glycolate, polyvinylpyrrolidone, polyethylene glycol, and magnesium stearate. Combinations of such components can be utilized, and such components and other components used in conventional tablets are understood in the art.

As used herein, “diluents” may be inert substances added to increase the bulk of the composition to make a tablet a practical size for compression. As such, they may also be referred to as bulking agents. Commonly used diluents include, but are not limited to, microcrystalline cellulose (MCC), wood cellulose, corn starch, modified corn starch, (tri)calcium phosphate, calcium sulfate, lactose, kaolin, mannitol, sodium chloride, dry starch, (powdered) sugar, dextrose, mannitol, sorbitol, and the like. The diluent/bulking agent may be used alone or in various mixtures, and utilized in any amount known in the art for oral compositions.

As used herein, “flavoring agents” are compounds designed to give the composition a more palatable taste. Flavoring agents vary considerably in their chemical structure, ranging from simple esters, alcohols, and aldehydes to carbohydrates and complex volatile oils. Synthetic flavors of almost any desired type are now available and are well known in the art. If hard taste, acid taste or bitter taste derived from starting materials may be suppressed by seasoning or flavoring, the acidulant (e.g., citric acid, tartaric acid, malic acid, ascorbic acid, etc.), the sweetener (e.g. sodium saccharin, dipotassium glycyrrhizinate, aspartame, stevia, thaumatin, etc.), or the perfume (e.g. various fruit perfumes containing lemon oil, orange oil or strawberry, and yoghurt, mint, menthol, etc.) may be included in the composition. The flavoring agent may be used alone or in various mixtures, and utilized in any amount known in the art for oral compositions.

As used herein, “lubricants” are materials that perform a number of functions relating to compositions. In certain embodiments, like tablet manufacture, the lubricants perform one or more functions such as improving the rate of flow of the tablet granulation, preventing adhesion of the tablet material to the surface of dies and punches, reducing interparticle friction, and facilitating the ejection of the tablets from a die cavity. Examples of suitable lubricants include, but are not limited to, zinc stearate, gum arabic powder, cacao butter, carnauba wax, carmellosecalcium, carmellosesodium, caropeptide, aqueous silicon dioxide, dried aluminum hydroxide gel, glycerin, magnesium silicate, light anhydrous silicic acid, light liquid paraffin, crystalline cellulose, hardened oil, synthetic aluminum silicate, sesame oil, flour starch, white beeswax, magnesium oxide, dimethyl polysiloxane, potassium sodium tartrate, sucrose fatty acid ester, glycerin fatty acid ester, silicon resin, aluminum hydroxide gel, stearyl alcohol, stearic acid, aluminum stearate, calcium stearate, polyoxyl stearate, magnesium stearate, cetanol, gelatin, talc, magnesium carbonate, precipitated calcium carbonate, cornstarch, lactose, hard fat, saccharose, potato starch, hydroxypropylcellulose, fumaric acid, sodium stearyl fumarate, polyethylene glycol, polyoxyethylene polyoxypropylene glycol, polysorbate, beeswax, magnesium aluminometasilicate, methylcellulose, Japan wax, glycerin monostearate, sodium lauryl sulfate, calcium sulfate, magnesium sulfate, liquid paraffin, phosphoric acid, palmitic acid, and hydrogenated vegetable oils and fats. The lubricant may be used alone or in various mixtures, and utilized in any amount known in the art for oral compositions.

As used herein, “binders” are agents used to impart cohesive qualities to powdered materials. Binders, or “granulators” as they are sometimes known, impart a cohesiveness to the tablet formulation, which ensures the tablet remaining intact after compression, as well as improving the free-flowing qualities by the formulation of granules of desired hardness and size.

Materials commonly used as binders include starch, such as corn starch and pregelatinized starch; gelatin; sugars, such as sucrose, glucose, dextrose, molasses, and lactose; natural and synthetic gums, such as gum acacia, sodium alginate, extract of Irish moss, panwar gum, ghatti gum, mucilage of isapol husks, carboxymethylcellulose, methylcellulose, polyvinylpyrrolidone (PVP), Veegum, microcrystalline cellulose, microcrystalline dextrose, amylose, larch arabogalactan, ethyl cellulose, cellulose acetate, and the like. The binder may be used alone or in various mixtures, and utilized in any amount known in the art for oral compositions.

As used herein, “colorants” are agents that give the composition a more pleasing appearance, and in addition help the manufacturer to control the product during its preparation and help the user to identify the product. Any of the approved certified water-soluble FD&C dyes, mixtures thereof, or their corresponding lakes may be used to color tablets. A color lake is the combination by adsorption of a water-soluble dye to a hydrous oxide of a heavy metal, resulting in an insoluble form of the dye. The colorant may be used alone or in various mixtures, and utilized in any amount known in the art for oral compositions.

Other conventional ingredients that may optionally be present in the composition include preservatives, stabilizers, anti-adherents or silica flow conditioners or glidants, such as silicon dioxide. Such ingredients may be used alone or in various mixtures, and utilized in any amount known in the art for oral compositions.

It is to be appreciated that certain components or additives may be classified under different terms of art and just because a component or additive is classified under such a term does not mean that they are limited to that function. If utilized, the additive or additives may be present in the composition in various amounts. Additional ingredients for optional use in the composition, e.g. when adapted for topical or oral administration, are described in U.S. Pat. Nos. 5,747,006; 5,980,904; 6,994,874; 7,060,304; 7,247,321; 7,348,034; 7,364,759; 7,700,110; 7,722,904; 8,202,556; 8,916,212; 9,445,975; 9,801,809; 10,307,366; 10,532,024; and 10,537,516; and in U.S. Publication Nos. 2006/0257509; 2007/0224154; 2008/0081082; 2008/0124409; 2013/0302265; 2017/0252293; 2017/0281666; 2018/0200285; 2019/0083566; 2019/0160117; 2020/0171117; 2020/0383898; 2021/0017240; and 2021/0212926; the disclosures of which are hereby incorporated by reference in their entirety.

### Method of Administration

The composition may be administered or applied as needed, daily, several times per day or in any suitable regimen such that the desired outcome is achieved. In the method of this disclosure, the frequency of administration (e.g. topical application) can depend on several factors, including the desired level of elastase inhibition. Generally, a regimen includes application of the composition once or twice daily to include an administration in the morning and/or an administration in the evening. The amount and/or frequency of application of the composition may depend on several factors, including the level of desired results and the specific composition.

Improved skin appearance can be achieved by administering the formulations of the present invention externally, internally, or some combination thereof. Preferably, the formulations of the present invention are administered with an acceptable carrier. For example, the formulation of the present invention could be externally administered with an acceptable carrier in the form of a gel, lotion, cream, tonic, emulsion, etc. As a further example, the formulation of the present invention could be internally administered with an acceptable carrier in the form of a pill, tablet, powder, bar, beverage, etc. Thus, the formulations described herein are useful in a wide variety of finished products, including pharmaceutical products, food products, and beverage compositions. Preferably, the products are useful for providing mammalian skin with an improved appearance.

When the formulations of the present invention are orally administered in the form of a liquid, the liquid may be water-based, milk-based, tea-based, fruit juice-based, or some combination thereof. Solid and liquid formulations for internal administration according to the present invention can further comprise thickeners, including xanthan gum, carboxymethyl-cellulose, carboxyethylcellulose, hydroxypropylcellulose, methylcellulose, microcrystalline cellulose, starches, dextrins, fermented whey, tofu, maltodextrins, polyols, including sugar alcohols (e.g., sorbitol and mannitol), carbohydrates (e.g. lactose), propylene glycol alginate, gellan gum, guar, pectin, tragacanth gum, gum acacia, locust bean gum, gum arabic, gelatin, as well as mixtures of these thickeners. These thickeners are typically included in the formulations of the present invention at levels up to about 0.1%, depending on the particular thickener involved and the viscosity effects desired.

The solid and liquid (food and beverage) formulations of the present invention can, and typically will, contain an effective amount of one or more sweeteners, including carbohydrate sweeteners and natural and/or artificial no/low calorie sweeteners. The amount of the sweetener used in the formulations of the present invention will vary, but typically depends on the type of sweetener used and the sweetness intensity desired.

In another example, the formulations of the present invention are topically administered in the form of a: solution, gel, lotion, cream, ointment, oil-in-water emulsion, water-in-oil emulsion, stick, spray, paste, mousse, tonic, foundation, mask, or other cosmetically and topically suitable form.

Preferably, formulations of the present invention that are suitable for topical administration are mixed with an acceptable carrier. An acceptable carrier may act variously as solvent, carrier, diluent or dispersant for the constituents of the composition, and allows for the uniform application of the constituents to the surface of the skin at an appropriate dilution. The acceptable carrier may also facilitate penetration of the composition into the skin.

In one example of a formulation for topical application, the acceptable carrier forms from about 70% to about 99.99% by weight of the total composition. In other examples, the acceptable carrier will form from about 85% to 99.99% by weight of the total composition. The acceptable carrier may also form from about 90% to about 99.99% by weight of the total composition; or from about 99.95% to about 99.999% by weight of the total composition. The acceptable carrier can, in the absence of other cosmetic adjuncts or additives, form the balance of the composition.

The various ingredients used in practicing the present invention may be soluble or insoluble in the acceptable carrier. If all ingredients of a formulation are soluble in the acceptable carrier, then the vehicle acts as solvent. However, if all or some ingredients of a formulation are insoluble in the acceptable carrier, then those ingredients are dispersed in the vehicle by means of, for example, a suspension, emulsion, gel, cream or paste, and the like.

Thus, it will be apparent to the skilled artisan that the range of possible acceptable carriers is very broad. For example, acceptable carriers can be emulsions, lotions, creams, or tonics. Acceptable carriers can comprise water, ethanol, butylene glycol, or other various solvents that aid in penetration of the skin. Some examples of suitable vehicles are described in U.S. Pat. Nos. 6,184,247 and 6,579,516, the entire contents of which are incorporated herein by reference.

Preferably the acceptable carrier used in practicing the present invention comprises water and ethanol. Optionally, the acceptable carrier also contains butylene glycol. For example, the acceptable carrier can comprise 2-5% butylene glycol by weight of the composition. In practicing the present invention, preferably this acceptable carrier is mixed with a formulation of the present invention comprising 2% by weight of the total composition. In other examples, the acceptable carrier is mixed with a formulation of the present invention comprising 0.001% to 30% by weight of the total composition; 1% to 5% by weight of the total composition; 0.01% to 15% by weight of the total composition; or 0.5% to 1.0% by weight of the total composition.

In general, however, acceptable carriers according to the present invention may comprise, but are not limited to comprising, any of the following examples: water; castor oil; ethylene glycol monobutyl ether; diethylene glycol monoethyl ether; corn oil; dimethyl sulfoxide; ethylene glycol; isopropanol; soybean oil; glycerin; soluble collagen; safflower seed oil; meadowfoam seed oil; mineral oil; squalene; shea butter; borage oil; or rice bran oil; polyquaternium-10; methylparaben; PEG-8; disodium lauroamphodacetate; sodium trideceth sulfate; hexylene glycol; sodium methyl cocoyl taurate; tea-lauryl sulfate; lauryl betaine; sodium myristoyl sarcosinate; PEG-150 distearate; citric acid-anhydrous; sodium citrate-dihydrate; diazolidinyl urea; disodium EDTA; propylparaben; polysorbate 60; isopropyl palmitate; octyl palmitate; C12-15 alkyl benzoate; dipropylene glycol dibenzoate; PPG-15 stearyl ether benzoate; isododecane; isoeicosane; squalane; jojoba oil; dimethicone; glyceryl stearate; PEG-100 stearate; cetyl alcohol; butylene glycol; chlorphenesin; fragrance; polyacrylamide; C13-14 isoparaffin; Laureth-7; aloe vera powder; aloe vera gel, hydroxyethylacrylate; sodium acryloyldimethyl taurate copolymer; behenyl alcohol; tocopheryl acetate; isodecyl neopentanoate; glyceryl trioctanoate; cetearyl alcohol; cetearyl glucoside; chamomilla recutita flower extract; biosaccharide gum-1; pentadecalactone; dipropylene glycol; cyclomethicone; PEG/PPG-18/18 Dimethicone; cyclopentasiloxane; disteardimonium hectorite; SD alcohol 40; phenoxyethanol; ethylparaben; trimethylsiloxysilicate; triethoxycaprylysilane; micronized titanium dioxide; titanium dioxide; zinc oxide; iron oxides (yellow; red; black; etc.); caprylysilane; sodium chloride; diisopropyl dimer dilinoleate; aluminum hydroxide; stearic acid; polyethelene beads; C12-15 alkyl benzoate; acrylates/C10-30 alkyl acrylate; xanthan gum; sorbitan laurate; panthenol; petrolatum; isopropyl isostearate; dimethicone; arginine; phenoxyethanol; acryloyldimethyl taurate copolymer; isohexadecane; polysorbate 80; hydroxyethylacrylate; sodium acryloyldimethyl taurate copolymer; octinoxate (octyl methoxycinnimate); oxybenzone; dicaprylyl ether; isodecyl neopentanoate; cetearyl alcohol; cetearyl glucoside; benzyl alcohol; HDI/trimethylol hexyllactone crosspolymer; silica; isodecyl neopentanoate; coco-glucoside; C20-22 alkyl phosphate; C20-22 alcohols; palmitoyl proline; magnesium palmitoyl glutamate; sodium palmitoyl sarcosinate; C30-45 alkyl cetearyl crosspolymer; polyacrylate 13; polyisobutene; polysorbate 20; iodopropynyl butylcarbamate; sodium magnesium silicate; methyl gluceth-20; dimethly isosorbide; silica; SD alcohol 40-B; salicylic acid; ceteth-20; fragrance; or witch hazel.

Additionally, acceptable carriers used in the present invention may optionally comprise one or more humectants, including but not limited to: dibutyl phthalate; soluble collagen; sorbitol; or sodium 2-pyrrolidone-5-carboxylate. Other examples of humectants that maybe used in practicing the present invention can be found in the CFTA Cosmetic Ingredient Handbook, the relevant portions of which are incorporated herein by reference.

Additionally, acceptable carriers in the present invention may optionally comprise one or more emollients including but not limited to: butane-1,3-diol; cetyl palmitate; dimethylpolysiloxane; glyceryl monoricinoleate; glyceryl monostearate; isobutyl palmitate; isocetyl stearate; isopropyl palmitate; isopropyl stearate; butyl stearate; isopropyl laurate; hexyl laurate; decyl oleate; isopropyl myristate; lauryl lactate; octadecan-2-ol; caprylic triglyceride; capric triglyceride; polyethylene glycol; propane-1,2-diol; triethylene glycol; sesame oil; coconut oil; safflower oil; isoamyl laurate; nonoxynol-9; panthenol; hydrogenated vegetable oil; tocopheryl acetate; tocopheryl linoleate; allantoin; propylene glycol; arachis oil; castor oil; isostearic acid; palmitic acid; isopropyl linoleate; lauryl lactate; myristyl lactate; decyl oleate; or myristyl myristate. Other examples of emollients that may be used in practicing the present invention can be found in the CFTA Cosmetic Ingredient Handbook, the relevant portions of which are incorporated herein by reference.

Additionally, acceptable carriers used in the present invention may optionally comprise one or more penetration enhancers including but not limited to: pyrrolidones, for example 2-pyrrolidone; alcohols, such as ethanol; alkanols, such as decanol; glycols, such as propylene glycol, dipropylene glycol, butylene glycol; surfactants; or terpenes.

Other acceptable carriers that may be used in practicing the present invention will be apparent to those of skill in the art and are included within the scope of the present invention.

For example, an acceptable carrier can be a lotion that is topically applied. The lotion may comprise cabomer 981, water, glycerin, isopropyl myristate, mineral oil, shea butter, stearic acid, glycol stearate, cetyl alcohol, dimethicone, preservatives, tea, and various ingredients of the formulations of the present invention.

The formulations of the present invention may also contain various known and conventional cosmetic adjuvants so long as they do not detrimentally affect the desired skin improvement and moisturizing effects provided by the formulation. For example, a formulation of the present invention can further include one or more additives or other optional ingredients well known in the art, which can include but are not limited to fillers (e.g., solid, semi-solid, liquid, etc.); carriers; diluents; thickening agents; gelling agents; vitamins, retinoids, and retinols (e.g., vitamin B₃, vitamin A, etc.); pigments; fragrances; sunscreens and sunblocks; anti-oxidants and radical scavengers; organic hydroxy acids; exfoliants; skin conditioners; moisturizers; ceramides, pseudoceramides, phospholipids, sphingolipids, cholesterol, glucosamine, pharmaceutically acceptable penetrating agents (e.g., n-decylmethyl sulfoxide, lecithin organogels, tyrosine, lysine, etc.); preservatives; antimicrobial agents; amino acids such as proline, pyrrolidone carboxylic acid, its derivatives and salts, saccharide isomerate, panthenol, buffers together with a base such as triethanolamine or sodium hydroxide; waxes, such as beeswax, ozokerite wax, paraffin wax; plant extracts, such as Aloe Vera, cornflower, witch hazel, elderflower, or cucumber and combinations thereof. Other suitable additives and/or adjuncts are described in U.S. Pat. No. 6,184,247, the entire contents of which are incorporated herein by reference.

The formulation can include additional inactive ingredients, including, but not limited to surfactants, co-solvents, and excipients. Surfactants, such as hydrophilic and hydrophobic surfactants, can be included in the formulations. Particular surfactants can be used based on the on the overall composition of the formulation and the intended delivery of the formulation. Useful surfactants include polyethoxylated (PEG) fatty acids, PEG-fatty acid diesters, PEG-fatty acid mono- and di-ester mixtures, polyethylene glycol glycerol fatty acid esters, alcohol-oil transesterification products, polyglycerized fatty acids, propylene glycol fatty acid esters, mixtures of propylene glycol esters-glycerol esters, mono- and diglycerides, sterol and sterol derivatives, polyethylene glycol sorbitan fatty acid esters, polyethylene glycol alkyl ethers, polysaccharide esters, polyethylene glycol alkyl phenols, polyoxyethylene-polyoxypropylene block copolymers, sorbitan fatty acid esters, lower alcohol fatty acid esters, ionic surfactants, and mixtures thereof.

The formulations can also include co-solvents such as alcohols and polyols, polyethylene glycols ethers, amides, esters, other suitable co-solvents, and mixtures thereof. The formulations can also include excipients or additives such as sweeteners, flavorants, colorants, antioxidants, preservatives, chelating agents, viscomodulators, tonicifiers, odorants, opacifiers, suspending agents, binders, and mixtures thereof.

Generally, the formulations of the present invention are topically or orally administered at least on a daily basis for a period of time sufficient to bring about the desired level of improvement in skin appearance. Topical application or oral administration of the formulations of the invention may continue for any suitable period of time. More specifically, within a few hours to within a few days of the initial application or ingestion, a user may notice the skin has an improved appearance. It should be appreciated that the frequency with which the formulations of the present invention should be applied or ingested will vary depending on the desired level improved appearance. In particular, the degree of cosmetic enhancement will vary directly with the total amount of composition used.

Useful dosage forms can be prepared by methods and techniques that will be well understood by those of skill in the art and may include the use of additional ingredients in producing tablets, capsules, or liquid dosage forms.

### INDUSTRIAL APPLICABILITY

This disclosure provides new and useful elastase inhibitors, including the botanical extracts described herein. Such elastase inhibitors can offer potential preventive and therapeutic approaches for lowering the risks of skin again, such as wrinkle formation, caused by elastase activity. As such, the compositions and methods described herein are useful for inhibiting elastase.

General compositions and product lines provided by this disclosure relate to skin care and nutrition-beauty products utilizing such elastase inhibitors, and specific examples include eye creams, face creams, and masks utilizing such elastase inhibitors.

Left unchecked, elastase can undesirably degrade elastic fibers such as elastin, reducing skin elasticity, increasing the appearance of wrinkles, and causing healing deficiencies. Thus, the compositions and methods herein are useful for elastase inhibition or skin aging prevention, and therefore can increase skin elasticity, decrease the appearance of wrinkles, and otherwise promote skin healing. The compositions and methods of this disclosure also provide potential preventive and therapeutic approaches for lowering the risks of skin aging caused by elastase activity.

The following examples, illustrating the compositions and methods of this disclosure, are intended to illustrate and not to limit the disclosure.

### Examples

The inhibition effects of six botanic ethanol and/or water extracts on elastase activity were evaluated. Specifically, three extract samples of Potentilla glabra leaf, three extract samples of Bombax malabaricum flower, three extract samples of Amomum villosum seed, three extract samples of Amomum kravanh fruit, and six extract samples of Cornus officinalis fruit were evaluated for their potential to inhibit elastase activity. The eighteen extracts showed inhibition as illustrated in the following Tables and Figures. The exacts were made in lab, but are also commercially available.

### Inhibition of Elastase

The elastase inhibitory activity of certain plant extracts was evaluated in vitro using Porcine pancreatic elastase (PPE) enzyme inhibitory assay as described by Cannel et al. (1988) with some modifications. The assay was performed in 0.2 M Tris-HCL buffer (pH 8.0). Porcine pancreatic elastase was dissolved to make a 100 unit stock solution in sterile water. The substrate N-Succinyl-Ala-Ala-Ala-p-nitroanilide (AAAPVN) was dissolved in buffer. The plant extracts were incubated with the enzyme for 25 minutes before adding the substrate to begin the reaction. The final reaction mixture (250 μL) contained buffer, 10 μg/mL AAAPVN, 0.001 units PPE, and 500 μg/mL of plant extract. EGCG was used as a positive control, and tris-HCl was used as the blank.

### Determination of Inhibition Rate

Maximum Velocities (Vmax) were taken at a wavelength of 410 nm for 25 minutes at 30 second intervals. Percentage inhibitions were calculated by the following equation:

$$Inhibition rate ( \% ) = \frac{( V max ( control ) - V max ( sample ) )}{V max ( control )} \times 100 \%$$

where Vmax(sample) is the velocity of the sample extracts and Vmax(control) is the velocity of the assay using the buffer instead of inhibitor (sample).

The results are illustrated in the Tables below. The average rates of inhibition can also be appreciated with reference to the Figures.

TABLE 1
EGCG (Positive)

| | | | | | | | |
|---|---|---|---|---|---|---|---|
| Concentration | 2000 | 1000 | 500 | 250 | 125 | 62.5 | 31.25 |
| (μg/ml) | | | | | | | |
| Inhibition Rate | 52.32 | 49.48 | 45.20 | 45.23 | 39.69 | 20.43 | 6.76 |
| (%) | 52.75 | 47.89 | 41.64 | 41.13 | 35.13 | 14.78 | 1.06 |
| | 53.28 | 49.56 | 43.58 | 38.32 | 33.94 | 11.52 | 6.58 |
| Average % | 52.78 | 48.97 | 43.47 | 41.56 | 36.25 | 15.57 | 4.80 |
| inhibition* | | | | | | | |

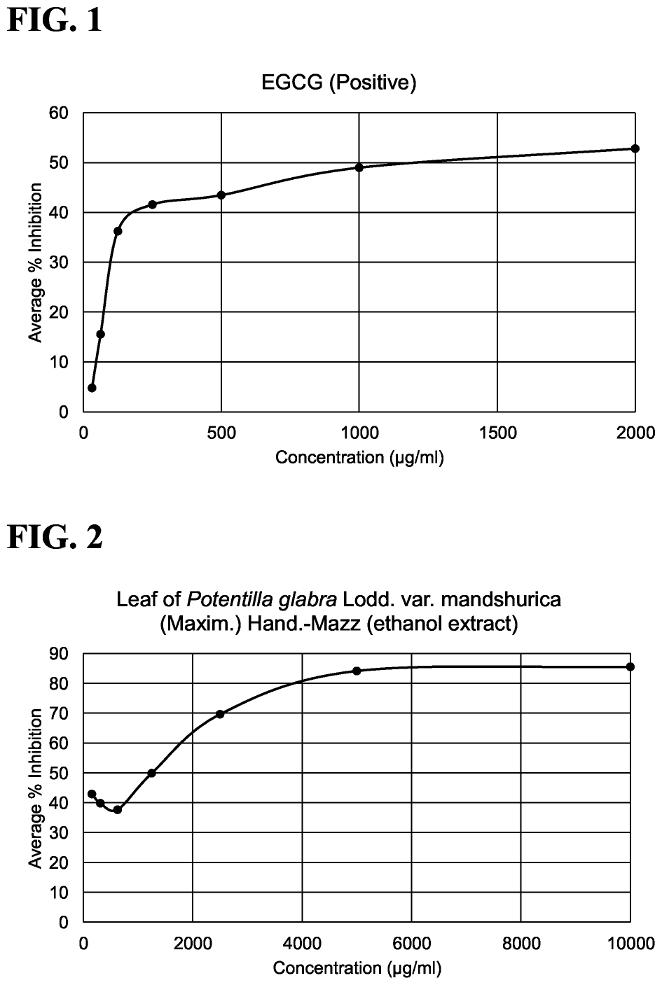

*see also FIG. 1 (where the X-axis is concentration and the Y-axis is average % inhibition)

TABLE 2
Leaf of Potentilla glabra Lodd. var. mandshurica
(Maxim.) Hand.-Mazz (ethanol extract)

| | | | | | | | |
|---|---|---|---|---|---|---|---|
| Concentration | 10000 | 5000 | 2500 | 1250 | 625 | 312.5 | 156.25 |
| (μg/ml) | | | | | | | |
| Inhibition Rate | 83.81 | 83.30 | 71.97 | 49.72 | 39.15 | 37.49 | 41.61 |
| (%) | 87.03 | 83.84 | 67.58 | 48.83 | 34.67 | 40.35 | 41.92 |
| | 85.87 | 85.31 | 69.39 | 50.97 | 39.06 | 41.45 | 45.07 |
| Average % | 85.57 | 84.15 | 69.65 | 49.84 | 37.63 | 39.76 | 42.86 |
| inhibition* | | | | | | | |

*see also FIG. 2 (where the X-axis is concentration and the Y-axis is average % inhibition)

TABLE 3
Flower of Bombax malabaricum DC. (ethanol extract)

| | | | | | | | |
|---|---|---|---|---|---|---|---|
| Concentration | 10000 | 5000 | 2500 | 1250 | 625 | 312.5 | 156.25 |
| (μg/ml) | | | | | | | |
| Inhibition Rate | 79.22 | 62.85 | 30.64 | 13.50 | −1.64 | −3.85 | −3.16 |
| (%) | 78.28 | 61.86 | 32.52 | 15.35 | 10.28 | 6.00 | 3.81 |
| | 78.73 | 62.23 | 32.83 | 19.73 | 8.54 | 9.02 | 7.68 |
| Average % | 78.74 | 62.31 | 32.00 | 16.19 | 5.72 | 3.72 | 2.78 |
| inhibition* | | | | | | | |

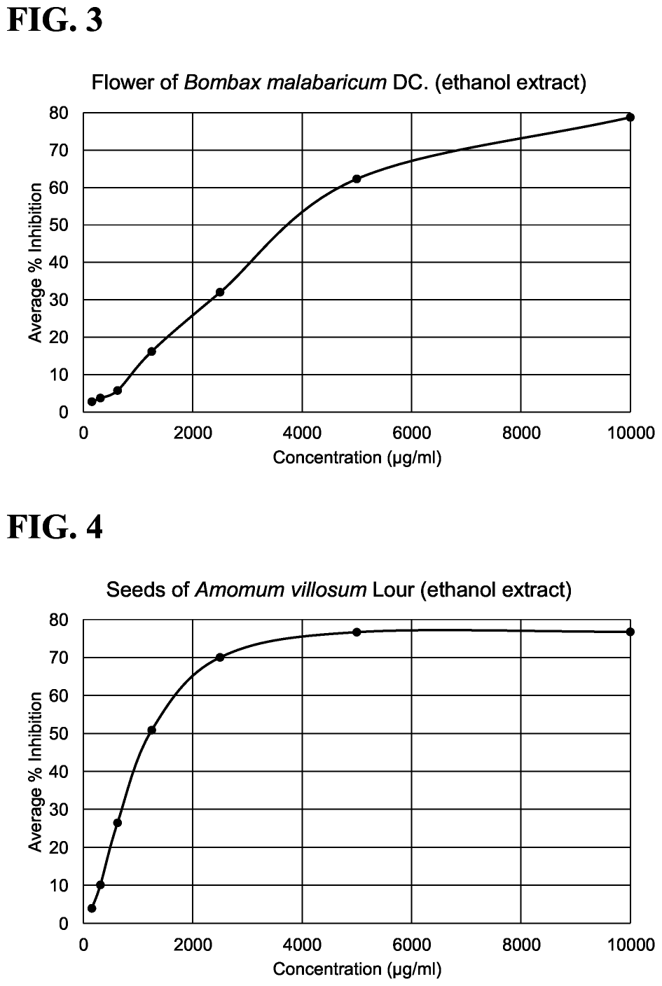

*see also FIG. 3 (where the X-axis is concentration and the Y-axis is average % inhibition)

TABLE 4
Seeds of Amomum villosum Lour (ethanol extract)

| | | | | | | | |
|---|---|---|---|---|---|---|---|
| Concentration | 10000 | 5000 | 2500 | 1250 | 625 | 312.5 | 156.25 |
| (μg/ml) | | | | | | | |
| Inhibition Rate | 75.56 | 76.34 | 69.77 | 49.84 | 24.69 | 6.56 | 0.73 |
| (%) | 77.09 | 75.92 | 69.46 | 49.33 | 22.35 | 8.27 | 0.98 |
| | 77.68 | 77.78 | 70.85 | 53.45 | 32.37 | 15.44 | 10.12 |
| Average % | 76.77 | 76.68 | 70.03 | 50.87 | 26.47 | 10.09 | 3.94 |
| inhibition* | | | | | | | |

*see also FIG. 4 (where the X-axis is concentration and the Y-axis is average % inhibition)

TABLE 5
Fruit of Amomum kravanh (ethanol extract)

| | | | | | | | |
|---|---|---|---|---|---|---|---|
| Concentration | 10000 | 5000 | 2500 | 1250 | 625 | 312.5 | 156.25 |
| (μg/ml) | | | | | | | |
| Inhibition Rate | 96.87 | 71.39 | 37.55 | 21.21 | 12.74 | 6.50 | 11.26 |
| (%) | 97.39 | 64.68 | 36.08 | 21.16 | 13.24 | 12.61 | 13.92 |
| | 96.08 | 66.79 | 39.11 | 24.61 | 16.68 | 20.84 | 23.92 |
| Average % | 96.78 | 67.62 | 37.58 | 22.33 | 14.22 | 13.32 | 16.37 |
| inhibition* | | | | | | | |

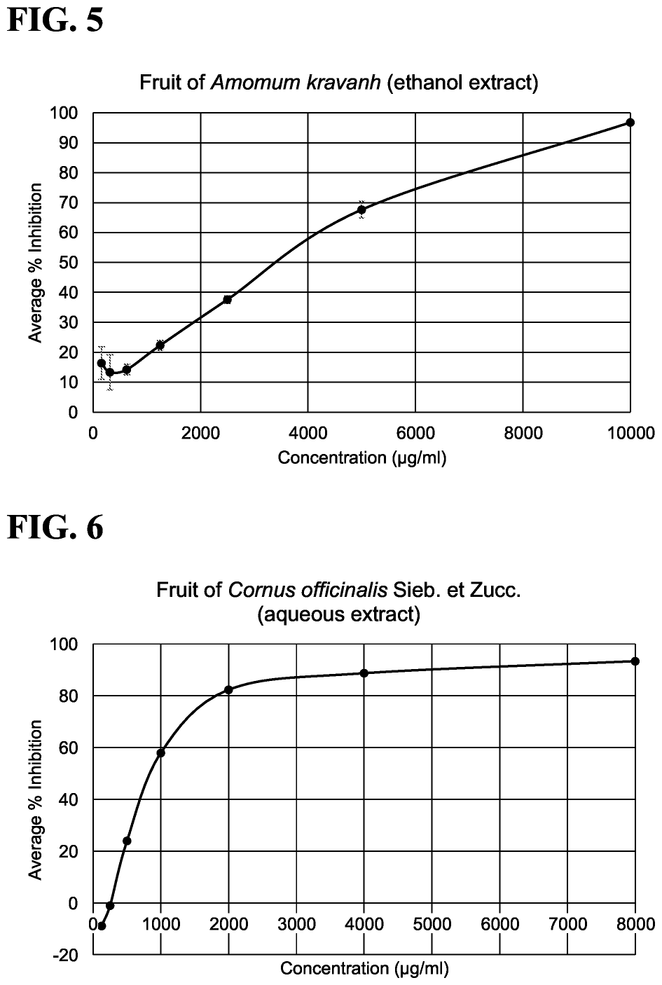

*see also FIG. 5 (where the X-axis is concentration and the Y-axis is average % inhibition)

TABLE 6
Fruit of Cornus officinalis Sieb. et Zucc. (aqueous extract)

| | | | | | | | |
|---|---|---|---|---|---|---|---|
| Concentration | 8000 | 4000 | 2000 | 1000 | 500 | 250 | 125 |
| (μg/ml) | | | | | | | |
| Inhibition Rate | 93.47 | 88.64 | 82.54 | 58.19 | 27.07 | 3.17 | −0.10 |
| (%) | 92.81 | 88.19 | 81.57 | 57.76 | 20.02 | −1.40 | −14.97 |
| | 93.69 | 89.26 | 82.77 | 57.78 | 24.70 | −5.07 | −11.71 |
| Average % | 93.32 | 88.70 | 82.29 | 57.91 | 23.93 | −1.10 | −8.93 |
| inhibition* | | | | | | | |

*see also FIG. 6 (where the X-axis is concentration and the Y-axis is average % inhibition)

TABLE 7
Fruit of Cornus officinalis Sieb. et Zucc. (ethanol extract)

| | | | | | | | |
|---|---|---|---|---|---|---|---|
| Concentration | 8000 | 4000 | 2000 | 1000 | 500 | 250 | 125 |
| (μg/ml) | | | | | | | |
| Inhibition Rate | 83.75 | 62.40 | 42.52 | 16.27 | 10.07 | 8.03 | 3.11 |
| (%) | 83.17 | 57.92 | 38.76 | 18.51 | 10.34 | 5.01 | 4.01 |
| | 81.76 | 52.22 | 29.83 | 10.97 | 2.48 | −0.51 | −1.37 |
| Average % | 82.89 | 57.51 | 37.04 | 15.25 | 7.63 | 4.18 | 1.92 |
| inhibition* | | | | | | | |

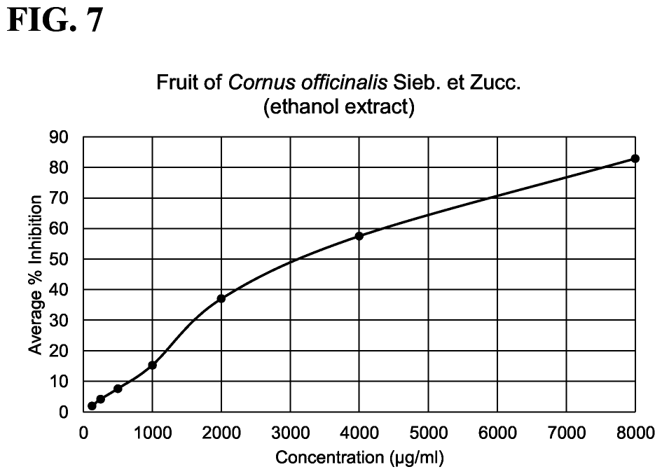

*see also FIG. 7 (where the X-axis is concentration and the Y-axis is average % inhibition)

### Additional Embodiments

The following additional embodiments are provided, the numbering of which is not to be construed as designating levels of importance.

Embodiment 1 relates to a composition for administration to a subject, the composition comprising at least one botanical active component, wherein the botanical active component comprises at least one extract selected from the group consisting of: i) an extract of Potentilla glabra leaf; ii) an extract of Bombax malabaricum flower; iii) an extract of Amomum villosum seed; iv) an extract of Amomum kravanh fruit; v) an extract of Cornus officinalis fruit; and vi) combinations of i) to v); wherein the botanical active component is present in the composition in an amount effective to inhibit elastase activity in skin of the subject.

Embodiment 2 relates to the composition according to Embodiment 1, wherein the botanical active component comprises the extract of Potentilla glabra leaf, optionally wherein the botanical active component consists of the extract of Potentilla glabra leaf.

Embodiment 3 relates to the composition according to Embodiment 1 or 2, wherein the extract of Potentilla glabra leaf is obtained by alcohol extracting, optionally ethanol extracting, leaves or leaf-based plant material of Potentilla glabra.

Embodiment 4 relates to the composition according to any one of Embodiments 1 to 3, wherein the composition is substantially to completely free of components obtained from non-leaf-based plant material of Potentilla glabra.

Embodiment 5 relates to the composition according to any one of Embodiments 1 to 4, wherein the botanical active component comprises the extract of Bombax malabaricum flower, optionally wherein the botanical active component consists of the extract of Bombax malabaricum flower.

Embodiment 6 relates to the composition according to any one of Embodiments 1 to 5, wherein the extract of Bombax malabaricum flower is obtained by alcohol extracting, optionally ethanol extracting, flowers or flower-based plant material of Bombax malabaricum.

Embodiment 7 relates to the composition according to any one of Embodiments 1 to 6, wherein the composition is substantially to completely free of components obtained from non-flower-based plant material of Bombax malabaricum.

Embodiment 8 relates to the composition according to any one of Embodiments 1 to 7, wherein the botanical active component comprises the extract of Amomum villosum seed, optionally wherein the botanical active component consists of the extract of Amomum villosum seed.

Embodiment 9 relates to the composition according to any one of Embodiments 1 to 8, wherein the extract of Amomum villosum seed is obtained by alcohol extracting, optionally ethanol extracting, seeds or seed-based plant material of Amomum villosum.

Embodiment 10 relates to the composition according to any one of Embodiments 1 to 9, wherein the composition is substantially to completely free of components obtained from non-seed-based plant material of Amomum villosum.

Embodiment 11 relates to the composition according to any one of Embodiments 1 to 10, wherein the botanical active component comprises the extract of Amomum kravanh fruit, optionally wherein the botanical active component consists of the extract of Amomum kravanh fruit.

Embodiment 12 relates to the composition according to any one of Embodiments 1 to 11, wherein the extract of Amomum kravanh fruit is obtained by alcohol extracting, optionally ethanol extracting, fruit or fruit-based plant material of Amomum kravanh.

Embodiment 13 relates to the composition according to any one of Embodiments 1 to 12, wherein the composition is substantially to completely free of components obtained from non-fruit-based plant material of Amomum kravanh.

Embodiment 14 relates to the composition according to any one of Embodiments 1 to 13, wherein the botanical active component comprises the extract of Cornus officinalis fruit, optionally wherein the extract of Cornus officinalis is further defined as an extract of Cornus officinalis fruit.

Embodiment 15 relates to the composition according to any one of Embodiments 1 to 14, wherein the extract of Cornus officinalis fruit is obtained by water extracting (or aqueous extracting) fruit or fruit-based plant material of Cornus officinalis.

Embodiment 16 relates to the composition according to any one of Embodiments 1 to 14, wherein the extract of Cornus officinalis fruit is obtained by alcohol extracting, optionally ethanol extracting, fruit or fruit-based plant material of Cornus officinalis.

Embodiment 17 relates to the composition according to any one of Embodiments 1 to 16, wherein the composition is substantially to completely free of components obtained from non-fruit-based plant material of Cornus officinalis.

Embodiment 18 relates to the composition according to any one of Embodiments 1 to 17, wherein the composition is further defined as a topical composition that is formulated for topical administration to the subject.

Embodiment 19 relates to the composition according to Embodiment 18, further comprising a cosmetically acceptable carrier, optionally wherein the cosmetically acceptable carrier is not naturally occurring.

Embodiment 20 relates to use of the composition according to any one of Embodiments 1 to 19, for inhibiting elastase activity in skin of a subject. Embodiment 20a relates to use of the composition according to any one of Embodiments 1 to 19 for the manufacture of a medicament for the treatment of skin, optionally for the treatment of elastase activity in skin of a subject.

Embodiment 20b relates to a medicament for treating skin, optionally for treating elastase activity in skin of a subject, the medicament comprising the composition according to any one of Embodiments 1 to 19.

Embodiment 21 relates to a method of inhibiting elastase activity in skin of a subject, the method comprising administering an effective amount of a composition to the subject, wherein the composition is according to any one of Embodiments 1 to 19.

Embodiment 22 relates to the method of Embodiment 21, wherein the composition is administered topically to the subject.

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples. The term “about” as used herein serves to reasonably encompass or describe minor variations in numerical values measured by instrumental analysis or as a result of sample handling. Such minor variations may be in the order of 0-10, +0-5, or +0-2.5, % of the numerical values. Further, the term “about” applies to both numerical values when associated with a range of values. Moreover, the term “about” may apply to numerical values even when not explicitly stated.

Generally, as used herein a hyphen “—” or dash “—” in a range of values is “to” or “through”; a “>” is “above” or “greater-than”; a “≥” is “at least” or “greater-than or equal to”; a “<” is “below” or “less-than”; and a “≤” is “at most” or “less-than or equal to.” On an individual basis, each of the aforementioned applications for patent, patents, and/or patent application publications, is expressly incorporated herein by reference in its entirety in one or more non-limiting embodiments.

It is to be understood that the appended claims are not limited to express and particular compounds, compositions, or methods described in the detailed description, which may vary between particular embodiments which fall within the scope of the appended claims. With respect to any Markush groups relied upon herein for describing particular features or aspects of various embodiments, it is to be appreciated that different, special, and/or unexpected results may be obtained from each member of the respective Markush group independent from all other Markush members. Each member of a Markush group may be relied upon individually and or in combination and provides adequate support for specific embodiments within the scope of the appended claims.

It is also to be understood that any ranges and subranges relied upon in describing various embodiments of the present invention independently and collectively fall within the scope of the appended claims, and are understood to describe and contemplate all ranges including whole and/or fractional values therein, even if such values are not expressly written herein. One of skill in the art readily recognizes that the enumerated ranges and subranges sufficiently describe and enable various embodiments of the present invention, and such ranges and subranges may be further delineated into relevant halves, thirds, quarters, fifths, and so on.

As just one example, a range “of from 0.1 to 0.9” may be further delineated into a lower third, i.e., from 0.1 to 0.3, a middle third, i.e., from 0.4 to 0.6, and an upper third, i.e., from 0.7 to 0.9, which individually and collectively are within the scope of the appended claims, and may be relied upon individually and/or collectively and provide adequate support for specific embodiments within the scope of the appended claims. In addition, with respect to the language which defines or modifies a range, such as “at least,” “greater than,” “less than,” “no more than,” and the like, it is to be understood that such language includes subranges and/or an upper or lower limit. As another example, a range of “at least 10” inherently includes a subrange of from at least 10 to 35, a subrange of from at least 10 to 25, a subrange of from 25 to 35, and so on, and each subrange may be relied upon individually and/or collectively and provides adequate support for specific embodiments within the scope of the appended claims. Finally, an individual number within a disclosed range may be relied upon and provides adequate support for specific embodiments within the scope of the appended claims. For example, a range “of from 1 to 9” includes various individual integers, such as 3, as well as individual numbers including a decimal point (or fraction), such as 4.1, which may be relied upon and provide adequate support for specific embodiments within the scope of the appended claims.

The present invention has been described herein in an illustrative manner, and it is to be understood that the terminology which has been used is intended to be in the nature of words of description rather than of limitation. Many modifications and variations of the present invention are possible in light of the above teachings. The present invention may be practiced otherwise than as specifically described within the scope of the appended claims. The subject matter of all combinations of independent and dependent claims, both single and multiple dependent, is herein expressly contemplated.

1. A composition for administration to a subject, the composition comprising at least one botanical active component, wherein the botanical active component comprises at least one extract selected from the group consisting of:
i) an extract of Potentilla glabra leaf;
ii) an extract of Bombax malabaricum flower;
iii) an extract of Amomum villosum seed;
iv) an extract of Amomum kravanh fruit;
v) an extract of Cornus officinalis fruit; and
vi) combinations of i) to v);
wherein the botanical active component is present in the composition in an amount effective to inhibit elastase activity in skin of the subject.

2. The composition according to claim 1, wherein the botanical active component comprises the extract of Potentilla glabra leaf, optionally wherein the botanical active component consists of the extract of Potentilla glabra leaf.

3. The composition according to claim 1, wherein the extract of Potentilla glabra leaf is obtained by alcohol extracting, optionally ethanol extracting, leaves or leaf-based plant material of Potentilla glabra.

4. The composition according to claim 1, wherein the composition is substantially to completely free of components obtained from non-leaf-based plant material of Potentilla glabra.

5. The composition according to claim 1, wherein the botanical active component comprises the extract of Bombax malabaricum flower, optionally wherein the botanical active component consists of the extract of Bombax malabaricum flower.

6. The composition according to claim 1, wherein the extract of Bombax malabaricum flower is obtained by alcohol extracting, optionally ethanol extracting, flowers or flower-based plant material of Bombax malabaricum.

7. The composition according to claim 1, wherein the composition is substantially to completely free of components obtained from non-flower-based plant material of Bombax malabaricum.

8. The composition according to claim 1, wherein the botanical active component comprises the extract of Amomum villosum seed, optionally wherein the botanical active component consists of the extract of Amomum villosum seed.

9. The composition according to claim 1, wherein the extract of Amomum villosum seed is obtained by alcohol extracting, optionally ethanol extracting, seeds or seed-based plant material of Amomum villosum.

10. The composition according to claim 1, wherein the composition is substantially to completely free of components obtained from non-seed-based plant material of Amomum villosum.

11. The composition according to claim 1, wherein the botanical active component comprises the extract of Amomum kravanh fruit, optionally wherein the botanical active component consists of the extract of Amomum kravanh fruit.

12. The composition according to claim 1, wherein the extract of Amomum kravanh fruit is obtained by alcohol extracting, optionally ethanol extracting, fruit or fruit-based plant material of Amomum kravanh.

13. The composition according to claim 1, wherein the composition is substantially to completely free of components obtained from non-fruit-based plant material of Amomum kravanh.

14. The composition according to claim 1, wherein the botanical active component comprises the extract of Cornus officinalis fruit, optionally wherein the extract of Cornus officinalis is further defined as an extract of Cornus officinalis fruit.

15. The composition according to claim 1, wherein the extract of Cornus officinalis fruit is obtained by water extracting (or aqueous extracting) fruit or fruit-based plant material of Cornus officinalis.

16. The composition according to claim 1, wherein the extract of Cornus officinalis fruit is obtained by alcohol extracting, optionally ethanol extracting, fruit or fruit-based plant material of Cornus officinalis.

17. The composition according to claim 1, wherein the composition is substantially to completely free of components obtained from non-fruit-based plant material of Cornus officinalis.

18. The composition according to claim 1, wherein the composition is further defined as a topical composition that is formulated for topical administration to the subject.

19. The composition according to claim 18, further comprising a cosmetically acceptable carrier, optionally wherein the cosmetically acceptable carrier is not naturally occurring.

20. (canceled)

21. A method of inhibiting elastase activity in skin of a subject, the method comprising administering, optionally topically administering, an effective amount of a composition to the subject, wherein the composition is according to claim 1.

22. (canceled)
